(19) United States
(12) Patent Application Publication
(10) Pub. No.: US 2023/0404880 A1
(43) Pub. Date: Dec. 21, 2023

(54) QUICK DISSOLVING, MILD, NATURAL AND SUSTAINABLE POWDER CLEANSING COMPOSITIONS FOR PERSONAL CARE FORMULATIONS

(71) Applicant: GALAXY SURFACTANTS LTD., Navi Mumbai (IN)

(72) Inventors: Pooja Vaidya, Nagpur (IN); Nirmal Koshti, Piscataway, NJ (US); Bharat Abhichandani, Pune (IN); Sneha S. Ghadigaonkar, Thane (IN)

(21) Appl. No.: 18/332,917

(22) Filed: Jun. 12, 2023

(30) Foreign Application Priority Data
Jun. 14, 2022 (IN) 202221034045

(51) Int. Cl. A61K 8/44; A61K 8/46; A61K 8/36; A61Q 19/10; A61Q 5/02; A61K 8/02

(52) U.S. Cl. CPC A61K 8/44; A61K 8/466; A61K 8/36; A61Q 19/10; A61K 2800/596; A61K 8/022; A61K 2800/42; A61K 2800/524; A61Q 5/02

(57) ABSTRACT

The present invention relates to mild, natural and sustainable powder cleansing composition which dissolves quickly in water providing excellent process ease to the consumer, to form a ready to use, homogenous personal care cleansing formulations. The personal care cleansing compositions of the present invention comprising blend of N-acyl glutamates and O-acyl isethionates, one or more co-surfactants, suspending agent and organic acid and when shaken with water at room temperature by the consumer, quickly dissolves over a period of less than 15 seconds to form ready to use cleansing compositions.

### FIELD OF INVENTION

The present invention relates to mild, natural and sustainable powder cleansing composition which dissolves quickly in water providing excellent process ease to the consumer, when mixed with water to form a ready to use, homogenous personal care formulations.

### BACKGROUND AND THE PRIOR ART

Personal care industry has come a long way in past few decades. Nowadays there is paradigm shift in consumer's need. In addition to efficient cleansing, consumer has lots of other expectations from the products such as aesthetics, mildness, ease of use. Today's consumers are well educated and want to avoid the use of products harmful to human health and the environment which has led to the need of natural and biodegradable formulations.

Various cleansing formulations like body washes, shampoos, face washes etc. contain more than 70% of water with maximum active content around 30%. Presence of high concentration of water helps in providing ease to use such formulations to the consumers but on the other hand leading to high carbon footprints. These diluted formulations are low viscous and therefore require proper packaging. More than 95% personal cleansing formulations are packed in plastic bottles leading to the concern of plastic recycling or wastage.

Though water appears plentiful globally, it is known that only 3% of it is available as fresh water on Earth, only 0.4% is available for usage as the rest is tied up in glaciers, snowpacks and ice caps. As per expert's opinion if significant responsible changes are not made in water consumption, two-third of the world may be facing water stress by the year 2025. Also, with regards to plastics, it is estimated that only 9% of all plastics produced is recycled, the rest gets dumped in landfills and oceans resulting in pollution, causing harm to birds, marine, mammal and fish.

To overcome these issues personal care industries are considering development of direct to use solid formulations as mentioned in WO2016/193938 which will help in reducing the use of water. Hence various direct to use formats like powder, granules, flakes and shampoo bars were considered for development. However, such formats have very less acceptance in the market as direct to use solid products do not provide ease of use and are still considered as inconvenient forms for consumers.

Further, in addition to be an efficient cleanser, consumer expects cleansing system to be mild to skin, hair and various surfaces. Hence, mild cleansing is one of the growing needs of personal care industry. Mild surfactants which perform cleansing without disturbing the hydration level of the skin are increasingly preferred by formulators. Furthermore, it should not disturb natural moisturizing factor (NMF), lipids and protein from the skin. In a nutshell, surfactant system must be a gentle cleanser and should respect the skin barrier integrity. In addition to cleansing and mildness, surfactant is expected to provide superior foaming and sensory properties. Naturalness of surfactants is another aspect which is keenly focused upon. All these requirements are globally well appreciated.

Therefore, it is challenging for formulators to develop powder cleansing compositions which in addition to being mild, natural, and sustainable also quickly dissolve in water and provides excellent process ease thereby excellent formulation experience to the consumers when added in water at the time of preparation.

GB 2523337 discloses dry powder cleansing compositions which focusses on composition which can be pre-weighed and mixed with water at the time of use. But the compositions are harsh as they are based on sodium lauryl ether sulfate and are not mild, natural, and sustainable. The compositions are not in the form of free-flowing powder and need mechanical operation for dissolving in water.

Choice of surfactants available in powder form is limited and mostly revolves around commonly used sulfates surfactants like sodium lauryl sulfates or sulfonate and lauryl sulfosuccinates.

IN 201821030525 discloses dry powder cleansing compositions based on combination of sodium lauryl sulfate and sulfosuccinate. Based on the powder cleansing composition of the patent application, Godrej has launched a hand wash which can be diluted with water at the consumer's end to form a ready to use aqueous product. The composition is based on combination of sodium lauryl sulfate and sulfosuccinate. But the composition does not dissolve quickly in the water. It remains non-homogenous, lumpy and takes more than 2 hours to form a homogenous liquid formulation. This results in hampering the process ease and hassle-free formulation experience to the consumers. Moreover, the composition is not mild, operates at pH higher than skin pH (around 6.5 to 7), hence resulting in lot of dryness on the skin post use.

In the attempt towards replacing harsh surfactants, sodium cocoyl isethionates are being used widely. Sodium cocoyl isethionate is a mild surfactant which is known for its moisturizing property and superior sensory. However, there is difficulty in dissolution of sodium cocoyl isethionate as most of the available forms of it are insoluble in water and require high temperature to form a homogenous dispersion in water. The requirement of such high temperature operations on industrial scale where the batch sizes run in metric tons are not always feasible. Hence, there are limitations for using this surfactant in the aqueous formulations also in the powder cleansing formulation where easy dissolution is prerequisite.

This limitation of using individual surfactant was suitably removed by a process wherein the powder blend of N-acyl glutamate and O-acyl isethionates is synthesized from more than one equivalence of same fatty acid chloride by reacting with alkali metal isethionate first and then with glycine in a sequential manner as described in the U.S. Pat. No. 9,308,156 by Koshti et al. Using this N-acyl glutamate and O-acyl isethionates; aqueous, high active, self-preserving compositions were created by Wankhade et al (U.S. Pat. No. 10,376,456 B2) but the invention was limited to aqueous formulations only.

Towards being responsible for the environment, reducing the usage of plastic and water and reducing carbon footprint, creation of powder to liquid compositions plays a vital role. There is a need to invent such quick dissolving powder cleansing compositions which are mild to skin and perform without compromising cleansing action and giving excellent processing experience to the consumer at the time of preparation.

It is evident from the prior art that the powder cleansing compositions available in the market are not mild, are very low at the sustainability index and are not quick dissolving so as to meet the mildness criteria and excellent/superior experience to the consumers. Hence, there is a need of powder cleansing compositions which dissolve quickly in water with dissolution time of less than 15 seconds, to form a ready to use, homogenous personal care cleansing formulations.

### OBJECTIVES OF THE INVENTION

1. It is an objective of the present invention to prepare powder cleansing compositions for personal care formulations that dissolve quickly in less than 15 seconds in water to form a homogenous formulation.
2. It is an objective of the present invention to prepare quick dissolving powder cleansing compositions for personal care formulations which requires no mechanical stirring at consumer's end at the time of dissolving and preparing the final product.
3. It is an objective of the present invention to prepare mild, natural and sustainable powder cleansing compositions for personal care formulations that provide superior process ease to the consumer.
4. It is an objective of the present invention to prepare powder cleansing compositions for personal care formulations that help in reducing water wastage and excessive usage of plastic.
5. Yet another objective of the present invention to prepare powder cleansing compositions for personal care formulations to provide effective cleansing without affecting the natural moisturization mechanism of skin or scalp/without solubilizing excessive proteins or lipids from the skin or scalp.

### SUMMARY OF THE INVENTION

Accordingly, the present invention provides a mild, natural and sustainable powder cleansing composition comprising blend of N-acyl glutamates and O-acyl isethionates, one or more co-surfactants, suspending agent and organic acid, such that said composition dissolves in water in less than 15 seconds to form homogenous personal care cleansing formulation.

In an aspect, the present invention relates to powder cleansing compositions which are easily processable at consumer's end when mixed with water at room temperature to form homogenous personal care cleansing compositions. The powder cleansing composition of the present invention are mild, natural and sustainable which dissolves quickly in water i.e., less than 15 seconds bringing lots of ease and excellent experience to the consumers at the time of dissolving.

The powder compositions of the present invention perfectly address the need of the marketplace to provide waterless, solid cleansing compositions which are easily processable at consumer's end when mixed with water to form personal cleansing products like handwash, body wash, face wash etc.

Powder cleansing compositions of the present invention comprising of blend of N-acyl glutamates and O-acyl isethionates, one or more cosurfactants, suspending agent and organic acid when combined with water quickly dissolves over a period of less than 15 seconds to form homogenous personal care cleansing formulations like hand wash, body wash, face wash, shampoo, conditioner, shaving foam etc.

Powder cleansing compositions of the present invention, comprise the blend of N-acyl glutamate of Formula I, and O-acyl isethionates of Formula II;

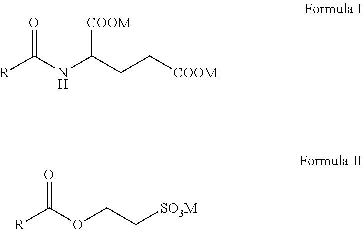

wherein,
R is selected from C₅ to C₂₂ alkyl group, saturated or unsaturated,
M is a cation selected from Li⁺, Na⁺, K⁺, NH₄⁺ or a quaternary ammonium cation derived from tertiary amines;
prepared by the process comprising steps of
A) reacting more than one equivalence of fatty acid chloride with alkali metal or ammonium salts of hydroxyalkyl sulphonates to prepare compounds of Formula II,
B) reacting the product of step (A) (containing the remainder fatty acid chloride) with glutamic acid in the presence of a base under typical aqueous Schotten Baumann reaction conditions to form compounds of Formula I

and is blended with one or more co-surfactants, suspending agent and organic acid.

The pH of the said powder cleansing compositions is 5.0 to 7.0.

### BRIEF DESCRIPTION OF ACCOMPANYING FIGURES

FIG. 1 illustrates the foam height of the powder hand wash cleansing compositions of the present invention (Example 2) and hand wash cleansing composition of comparative example 1A.

FIG. 2 illustrates the foaming properties of compositions of Example 3 and comparative example 3A.

FIG. 3 illustrates the scale for sensory performance.

FIG. 4 illustrates the comparison of sensory evaluation immediately after drying of hand wash cleansing composition of present invention (Example 2) and Godrej Protekt Mr. Magic Powder composition (Comparative Example 1B).

FIG. 5 illustrates the comparison of moisturization study of hand wash cleansing composition of present invention (Example 2) and Godrej Protekt Mr. Magic Powder composition (Comparative Example 1B).

FIG. 6 illustrates the comparison of mildness immediately after drying the hand wash cleansing composition of present invention (Example 2) and Godrej Protekt Mr. Magic Powder composition (Comparative Example 1B).

FIG. 7 illustrates the Zein score of the compositions of example 4 and comparative example 4A.

### DETAILED DESCRIPTION OF THE INVENTION

The present invention provides powder cleansing compositions that are prepared by the blend of N-acyl glutamates and O-acyl isethionates, one or more co-surfactants, suspending agent and organic acid. These powder compositions also help in reducing water wastage, avoiding the use of excessive plastic hence being ecofriendly and mild to skin without compromising cleansing action.

The inventors of the present invention have designed powder cleansing compositions comprising of the blend of N-acyl glutamate of Formula I, and O-acyl isethionates of Formula II;

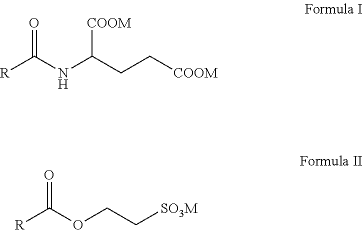

wherein,
R is selected from C₅ to C₂₂ alkyl group, saturated or unsaturated,
M is a cation selected from Li⁺, Na⁺, K⁺, NH₄⁺ or a quaternary ammonium cation derived from tertiary amines;
prepared by the process comprising steps of
A) reacting more than one equivalence of fatty acid chloride with alkali metal or ammonium salts of hydroxyalkyl sulphonates to prepare compounds of Formula II,
B) reacting the product of step (A) (containing the remainder fatty acid chloride) with glutamic acid in the presence of a base under typical aqueous Schotten Baumann reaction conditions to form compounds of Formula I.
and is blended with one or more co-surfactants, suspending agent and organic acid.

The pH of the said powder cleansing compositions is 5.0 to 7.0.

The blend of N-acyl glutamate of Formula I, and O-acyl isethionates of Formula II is present in the amount between 35 to 90% by weight.

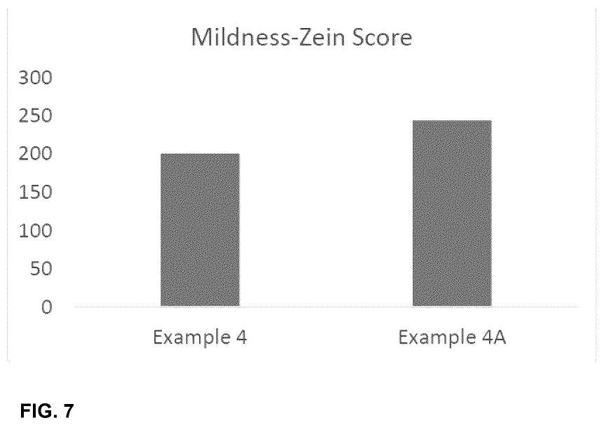

The inventors of the present invention have unexpectedly found that powder blends of N-acyl glutamate and O-acyl isethionates prepared as per the process of Koshti et. al with one or more cosurfactants, suspending agent and organic acid results in a powder cleansing composition which is mild, natural, sustainable and dissolves quickly in water to create various personal cleansing compositions. Thus, the present invention provides a novel quick dissolving powder cleansing compositions which are mild, natural and sustainable which when mixed with water at room temperature dissolves in less than 15 seconds to form a homogenous personal care cleansing formulation bringing lots of ease and excellent experience to the consumers at the time of dissolving. The composition of the present invention shows synergistic effect when N-acyl glutamate and O-acyl isethionate are combined with one or more co-surfactants. N-acyl glutamate and O-acyl isethionate along with co-surfactant, surprisingly shows enhanced foaming properties, as evident from FIG. 2. Also, the mildness of reconstituted powder composition is enhanced in presence of co-surfactant along with N-acyl glutamate and O-acyl isethionates when compared to reconstituted compositions that are devoid of co-surfactants. The same is evident from FIG. 7.

Gums and suspending agents are used in present invention to form gel composition or play role in building viscosity of personal care formulations and generally takes time to solubilize or tend to form agglomerate in such formulations. Suspending agents when used with blend of N-acyl glutamate and O-acyl isethionate along with organic acid surprisingly dissolves quickly and does not form any agglomerates during or post dissolution.

Accordingly, the present invention provides a powder cleansing composition comprising: blend of N-acyl glutamate of Formula I, and O-acyl isethionates of Formula II

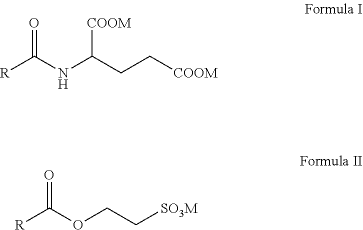

wherein,
R is selected from C₅ to C₂₂ alkyl group, saturated or unsaturated,
M is a cation selected from Li⁺, Na⁺, K⁺, NH₄⁺ or a quaternary ammonium cation derived from tertiary amines;
prepared by the process comprising steps of
A) reacting more than one equivalence of fatty acid chloride with alkali metal or ammonium salts of hydroxyalkyl sulphonates to prepare compounds of Formula II,
B) reacting the product of step (A) (containing the remainder fatty acid chloride) with glutamic acid in the presence of a base under typical aqueous Schotten Baumann reaction conditions to form compounds of Formula I.
   and
C) blending one or more surfactants, suspending agent and organic acids.

The novel powder cleansing compositions of the present invention possess high dissolution rates in water, providing excellent process ease to the consumers at the time of use. In addition to the above-mentioned properties, the powder cleansing compositions of the present invention, also have high % natural index and high % sustainability quotient.

The powder compositions of the present invention perfectly address the need of the marketplace to provide waterless, solid cleansing compositions which are easily processable at consumer's end when mixed with water to form homogenous personal care cleansing formulations like body wash, face wash, facial foam, hand wash, hand foam, shampoo, shower gel, shaving foam etc.

The co-surfactant(s) suitable for the present invention are selected from amphoteric, and anionic surfactants in addition to O-acyl isethionates and N-acyl glutamates, and mixtures thereof. These surfactants are explained in detail as follows.

### Amphoteric Surfactants

Amphoteric surfactants which can be used in this invention include at least one acid group. This may be a carboxylic or a sulphonic acid group. They include quaternary nitrogen and therefore are quaternary amido acids. They generally include an alkyl or alkenyl group of 7 to 18 carbon atoms and comply with general structural formula:

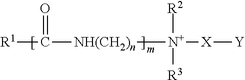

where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Examples of suitable amphoteric surfactants are alkyl betaines; amidoalkyl betaines; amphocarboxylate derivatives such as (mono or di) alkylamphoacetate; and amidoalkyl sultaines. Cocamidopropyl betaine commercially available as Galaxy CAPB SB, lauramidopropyl betaine commercially available as Galaxy LAPB, cocamidopropyl hydroxysultaine commercially available as Galaxy CAPSB, laurylamidopropyl hydroxysultaine, lauryl betaine, and lauryl hydroxysultaine, are particularly useful and preferred amphoteric surfactants for the present invention. If required coco-betaine commercially available as Galaxy CB can also be used in combination.

### Anionic Surfactants

Anionic surfactants which can be used in this invention include alkyl sulfate. Among the alkyl sulfates are those having the formula: ROSO₃M and wherein R is an alkyl or alkenyl having 8 to 18 carbons, preferably 12 to 18 carbons, and more preferably 12 to 14 carbons; and M is a cation such as sodium, potassium, ammonium or substituted ammonium ion. The preferred anionic surfactant is sodium lauryl sulfate.

Other anionic surfactants which can be used include aliphatic sulfonates, such as a primary alkane (e.g., C₈-C₂₂) sulfonates, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or an aromatic sulfonate such as alkyl benzene sulfonate.

Alkyl sulfosuccinates and ethoxylated alkyl sulfosuccinates (including mono- and dialkyl, e.g., C₆-C₂₂ sulfosuccinates) those can be used in the present invention are as disodium lauryl sulfosuccinate commercially available as Galaxy LSS.

Other anionic surfactants also include range of amino acid surfactants such N-acyl sarcosinates, N-acyl taurates, N-acyl glycinates N-acyl alaninates and surfactants like glucose carboxylates, alkyl ether carboxylates, alkyl amphoacetates, and mixture thereof.

The glycinate surfactant useful in present invention conforms generally to structural formula

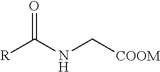

where R is alkyl or alkenyl of 5 to 22 carbon atoms or mixture of both, M is a cation selected from Li⁺, Na⁺, K⁺, NH₄⁺ or a quaternary ammonium cation derived from tertiary amines. Preferred N-acyl glycinates are sodium cocoyl glycinate or sodium lauroyl glycinate. One of the commercially available glycinate surfactant is with trade name Galsoft SCG.

The taurate surfactants useful in present invention are salts of fatty acid amides of N-methyltaurine. They conform generally to structural formula

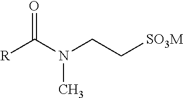

where R is alkyl or alkenyl of 5 to 22 carbon atoms or mixture of both, M is a cation selected from Li⁺, Na⁺, K⁺, NH₄⁺ or a quaternary ammonium cation derived from tertiary amines. The most preferred taurate are sodium methyl cocoyl taurate or sodium methyl lauroyl taurate. One of the commercially available taurate surfactant is with the trade name Galsoft SCT.

The sarcosinate surfactants useful in present invention are N-acyl sarcosinates which are obtained from reaction of fatty acid with sarcosine. They conform generally to structural formula

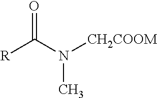

where R is alkyl or alkenyl of 5 to 22 carbon atoms or mixture of both, M is a cation selected from Li⁺, Na⁺, K⁺, NH₄⁺ or a quaternary ammonium cation derived from tertiary amines. The most preferred sarcosinate are sodium lauoryl sarcosinate and/or sodium cocoyl sarcosinate. One of the commercially available sarcosinate surfactant is with trade name Galsoft NaLS.

According to a preferred embodiment one or more cosurfactants are present in an amount greater than 5% by weight, preferably 5-45% by weight and are selected from N-acyl glycinate, N-acyl sarcosinate, N-acyl taurate, alkyl amidopropyl sulfobetaines, sulfosuccinates, alkyl betaines, alkyl amidopropyl betaines, alkyl sulfates and alkyl ether sulfates.

### Suspending Agents

Suspending agent suitable for the present invention is selected from long chain polymeric molecules like hydroxy ethyl cellulose, sodium carboxymethyl cellulose and hydroxypropylcellulose, hydroxypropylmethylcellulose. Also, other suitable suspending agents are natural gums like guar gum, gum acacia, xanthan gum, gum tragacanth and so on. The suspending agents are present in an amount between 3-11% by weight of the total composition.

The preferred suspending agents which can be part of compositions of present invention described herewith are from group of polymers from natural origin in chemically modified or unmodified forms. Polygalactomannas are widely used as suspending agents. These are polysaccharides composed principally of galactose and mannose units and are usually found in the endosperm of leguminous seeds such as guar, locust bean, honey locust, flame tree. The polygalactomannans may be used in either their natural form or may be substituted with one or more functional groups. The most commonly used polygalactomannan is guar gum. The present invention may also contain Xanthan gum which is a fermentation product prepared by action of the bacteria of the genus Xanthomonas upon carbohydrates. Four species of Xanthomonas, X. Campestris, X. phaseoli, X. malvocearum, and X. carotal are reported in the literature to be the most efficient gum producers. Although the exact chemical structure is not determined, it is generally accepted to be a heteropolysaccharide with a molecular weight of several million.

The suspending agents may be from group of synthetic and natural polymeric carbohydrates and derivatives. Examples of such materials include starch, modified starch, and mixtures.

### Organic Acids or Preservatives

Preservatives suitable for the present invention is selected from carboxylic acid (in free acid form) or their salts chosen from salts of benzoic acid, propionic acid, salicylic acid, sorbic acid, 4-hydroxybenzoic acid, dehydroacetic acid, formic acid, p-methoxybenzoic acid, acetic acid, or 10-undecylenic acid, lactic acid, undecenoic acid, glycolic acid, maleic acid, malic acid, citric acid, gluconic acid and their salts. The carboxylic acid component preferably comprises benzoic acid salts and/or sorbic acid salts, sodium benzoate and potassium sorbate being most preferred. The organic acid is present in an amount 4 to 12% by weight of the total composition.

### Optional Ingredients

In addition to the amphoteric and/or anionic surfactants, suspending agent and organic acids, the aqueous cleansing compositions of the present invention can optionally comprise of one or more cationic surfactants. Many cationic surfactants are known in the art and almost any of the cationic surfactants is suitable for optional use in the present invention.

The powder cleansing composition may contain various conditioning agents, fragrances, colour, benefit agents, and other personal care additives known in the art.

The powder cleansing composition may contain chelating agents like Ethylenediaminetetraacetic Acid or its Sodium salt, Disodium Etidronate, Sodium Gluconate, Tetrasodium glutamate diacetate.

The novel powder cleansing compositions of the present invention are natural and sustainable, quick dissolving in water hence providing excellent process ease to the consumer while making various personal cleansing formulations. The compositions are mild, foaming and capable of moisturization as well.

The performance characteristics of the present novel powder cleansing compositions are explained as below.

### Moisturization:

The powder cleansing compositions advantageously provide enhanced moisturization. Hand wash composition of the present invention (Example 2) shows a significant improvement in the hydration levels of skin as compared to market product Godrej Protekt Mr. Magic Powder composition.

### Sensory Benefits:

Sensory Evaluation is defined as “A scientific discipline used to evoke, measure, analyze, and interpret those responses to products that are perceived by the senses of sight, smell, touch, taste, and hearing (Stone and Sidel 1993).”

Unlike GB 2523337, novel compositions of present invention also provide excellent skin sensorial properties.

### Mildness:

Powder cleansing compositions of the current invention is extremely mild to skin and hair. It provides efficient cleansing without having any harmful effect on skin proteins and lipids. Example 2 demonstrates extremely mild behaviour of powder cleansing composition when compared with Godrej magic hand wash available in the market Example 1B.

### Foaming:

Despite the excellent moisturization properties of the powder cleansing compositions of the present invention, it also demonstrates good creamy lather. In general, the amount of foam generated by a cleansing composition is directly related to its perceived cleansing efficacy. The greater is the volume of foam produced and the greater is the stability of the foam; the more efficient is the perceived cleansing action. Surprisingly, it has been found that the powder cleansing compositions of the present invention exhibit better foam behaviour than the compositions mentioned in GB2523337 Murray et al. The presence of an excellent foaming effect in powder cleansing composition and its dilution was demonstrated by foam measurements (Hart De George method) in the experimental section.

### Advantages of the Invention

1. Novel Powder cleansing compositions of the present invention dissolve quickly in water in less than 15 seconds to form homogenous formulations.
2. The powder cleansing compositions of the present invention are quick dissolving with the help of hand shaking and doesn't require any equipment or mechanical stirring to form various personal cleansing formulations.
3. The powder cleansing composition of the present invention provides excellent formulation experience to the consumer when mixed with water to form ready to use aqueous cleansing compositions.
4. The novel powder cleaning compositions of the present invention are mild and sustainable which reduces water wastage and excessive use of plastic.
5. The novel powder cleansing compositions of the present invention have high naturality index which provide highly natural personal care cleansing composition having more than 70% naturalness as per ISO section 5.2.2 of ISO 16128-2.
6. Powder cleansing compositions of the present invention have very good foam profile, suitable personal care cleansing formulations.
7. The novel powder cleansing compositions of the present invention are prepared using ‘super mild’ surfactants and shows very good moisturization and sensorial properties on skin and hair.

### EXAMPLES

The present invention is now described by way of working non-limiting illustrative examples. These examples are provided for illustrative purposes only and are not intended to limit the scope of the invention.

Preservative: Sodium Benzoate Powder, procured from s-d fine-chem limited. Sodium Dehydroacetate Powder, procured from Connect Chemicals India Pvt Ltd.

pH Adjusting Ingredient: Citric Acid Powder procured from s-d fine-chem limited.

Suspending Agents: Sodium Carboxymethylcellulose with trade name Blanose CMC 9M31XF, hydroxyethylcellulose with trade name Natrosol 250 HHX, Hydroxypropyl Methylcellulose with trade name Benecel E10M, procured from Ashland Specialties.

Hair Conditioning Agent: Cetrimonium Chloride or Cetyltrimetylammonium chloride (25% active content) with trade name Varisoft 300 procured from Evonik Industries.

### Ingredients & Accessories Required for Making Powder Composition Mentioned Herein:

All the quantities are given in percentage (%) and mentions use of ingredients on as is basis i.e., considered purity, impurity and side product together in composition.

### Surfactants:

TABLE 1

| Product Trade Name | INCI Name | % active |
|---|---|---|
| Galsoft GLI 21 (P) | Disodium Cocoyl Glutamate and | 84% Active |
| | Sodium Cocoyl Isethionate | Powder |
| Galsoft SCT 40 | Sodium Methyl Cocoyl Taurate | 88% Active |
| | | Powder |
| Galsoft NaLS | Sodium Lauroyl Sarcosinate | 96% Active |
| | | Powder |
| Galsoft SLT (N) | Sodium Methyl Lauroyl Taurate | 88% Active |
| | | Powder |
| Galaxy LES 70 | Sodium Lauryl Ether Sulfate | 70% Active |
| | | Liquid |
| Galsoft SCGL | Disodium Cocoyl Glutamate | 73% Active |
| | | Powder |
| Galsoft SCI 80 | Sodium Cocoyl Isethionate | 80% Active |
| | | Powder |

The comparative examples 1A, 1B and 1C prove superior performance of the compositions of the present invention over market samples in terms of mildness, naturalness and ability of quick dissolving to form a homogenous formulation and providing pleasant experience of excellent process ease from the consumers.

### Comparative Example 1A: Hand Wash Cleansing Composition of GB 2523337

TABLE 2

| Ingredients | % (w/w) |
|---|---|
| Galaxy LES 70 | 37.5 |
| Sodium Bicarbonate | 12.5 |
| Sodium Chloride | 12.5 |
| Sodium Carbonate | 5.0 |
| Citric Acid | 15.0 |
| Urea | 12.5 |
| EDTA | 5.0 |

Procedure: All the ingredients of table 2 were blended in mentioned quantities in a bottom driven mixer blade. First dry ingredients were added and mixed in mixer followed by addition of liquid materials (Galaxy LES 70). The resultant mixture obtained is pasty mass with no consumer acceptable form.

In 97.85 g of water in hand wash bottle added 2.15 g of hand wash cleansing composition and shook for 5 seconds. The hand wash dilution prepared had undissolved content even after 5 minutes, thus offers poor dilution ease against the inventive compositions. pH of the above hand wash is 8.0 to 9.0.

### Comparative Example 1B: Hand Wash Based on Market Available Powder Composition (Godrej Protekt Mr. Magic Powder to Liquid Wash, IN 201821030525, Example 1)

This hand wash composition consists of sodium lauryl sulphate, guar gum, disodium lauryl sulfosuccinate, fragrance, cellulose gum, silicone dioxide, methyl paraben, disodium EDTA, aloe vera extract, neem extract.

In 95.7 g of water in hand wash bottle, added 4.3 g of market available powder composition and shook it for 5 seconds. The hand wash dilution prepared had undissolved content even after 5 minutes, thus offers poor dilution ease against the compositions of present invention. Further supplier recommends it to use after 1 hour for better results. The pH of hand wash is 6.5 to 7.0.

### Comparative Example 1C: Powder Hand Wash Cleansing Composition Using Galsoft SCGL & Galsoft SCI 80 Separately as Individual Ingredients

TABLE 3

| Ingredients | % (w/w) |
|---|---|
| Galsoft SCGL | 46.97 |
| Galsoft SCI 80 | 23.49 |
| Hydroxypropyl Methylcellulose | 8.88 |
| Sodium Benzoate | 11.11 |
| Citric Acid | 7.33 |
| Aloe Vera Powder | 2.22 |

Procedure: All the ingredients of Table 3 were blended in mentioned quantities in a bottom driven mixer blade. The resultant powder hand wash cleansing composition was compared against inventive composition mentioned in Example 2.

In 94.0 g of water in hand wash bottle added 6.0 g of powder composition and handshook it for 5 seconds. It was observed that current comparative hand wash composition of Example 1C has undissolved particles left when compared against inventive composition mentioned in Example 2, having similar composition of disodium cocoyl glutamate and sodium cocoyl isethionate processed and used in the form of Galsoft GLI 21 which gets homogenized and achieve uniformity in less than 15 seconds.

### Example 2: Powder Hand Wash Cleansing Composition

TABLE 4

| Ingredients | % (w/w) |
|---|---|
| Galsoft GLI 21 | 65.46 |
| Galsoft SLT (N) | 5.00 |
| Hydroxypropyl Methylcellulose | 8.88 |
| Sodium Benzoate | 11.11 |
| Citric Acid | 7.33 |
| Aloe Vera Powder | 2.22 |

Procedure: All the ingredients of table 4 were blended in mentioned quantities in a bottom driven mixer blade. The resultant mixture is ready to use Powder Hand Wash Cleansing Composition. The % naturality of the powder hand wash cleansing composition as per section 5.2.2 of ISO 16128-2. is 79.79%.

In 94.0 g of water in hand wash bottle added 6.0 g of powder composition and handshook it for 5 seconds to form a homogeneous, ready to use hand wash formulation. The dissolution time of powder cleansing composition is less than 15 seconds in water at room temperature. The hand wash formulation of example 2 has a pH 5.5 to 5.7, transparent in appearance and dispense in the form of creamy lather.

### Example 3 and Comparative Example 3A: Powder Facial Foam Cleansing Composition

TABLE 5

| | | Comparative |
|---|---|---|
| | Example 3 | Example 3A |
| Ingredients | % (w/w) | % (w/w) |
| Galsoft GLI 21 | 40.20 | 82.2 |
| Galsoft NaLS | 42.00 | 0 |
| Citric Acid | 7.80 | 7.80 |
| Sodium Benzoate | 10.00 | 10.00 |

### Procedure:

All ingredients of table 5 were blended/mixed in mentioned quantities in a bottom driven mixer blade. The resultant mixture is ready to use Powder Facial Foam Cleansing Composition. The % naturality of powder facial foam cleansing composition as per section 5.2.2 of ISO 16128-2 is 70.54.

In 98 g of water in facial foam bottle added 2.0 g of powder cleansing composition and hand shook it for 5 seconds to form a homogeneous, ready to use facial foam. The dissolution time of powder cleansing composition is less than 15 seconds in water at room temperature. The facial foam formulation has a pH 5.74 and is transparent in appearance and dispense in the form of creamy lather.

### Example 4 and Comparative Example 4A: Powder Mild & Moisturizing Shampoo Cleansing Composition

TABLE 6

| | | Comparative |
|---|---|---|
| | Example 4 | Example 4A |
| Ingredients | % (w/w) | % (w/w) |
| Galsoft GLI 21 | 67.05 | 72.05 |
| Galsoft SCT 40 | 05.00 | 0 |
| Sodium Carboxymethyl Cellulose | 11.69 | 11.69 |
| Sodium Dehydroacetate | 7.84 | 7.84 |
| Citric Acid | 6.92 | 6.92 |
| Cetyltrimonium Chloride | 1.50 | 1.50 |

### Procedure:

All the ingredients of table 6 were blended in mentioned quantities in a bottom driven mixer blade. Preferably solid ingredients were mixed first followed by liquid ingredient. The resultant mixture is ready to use Powder Mild & Moisturizing Shampoo Composition. The % naturality of mild & moisturizing cleansing shampoo composition as per section 5.2.2 of ISO 16128-2 is 79.51%.

In 93.5 g of water in a bottle added 6.5 g of powder cleansing composition. Handshook well for 5 seconds. The powder dissolved within 15 seconds to give homogeneous ready to use mild and moisturizing shampoo. The shampoo formulation is at pH 5.64 with excellent hair conditioning properties.

Final dilutions based on powder cleansing compositions of the present invention mentioned above are natural and have naturality index more than 95%.

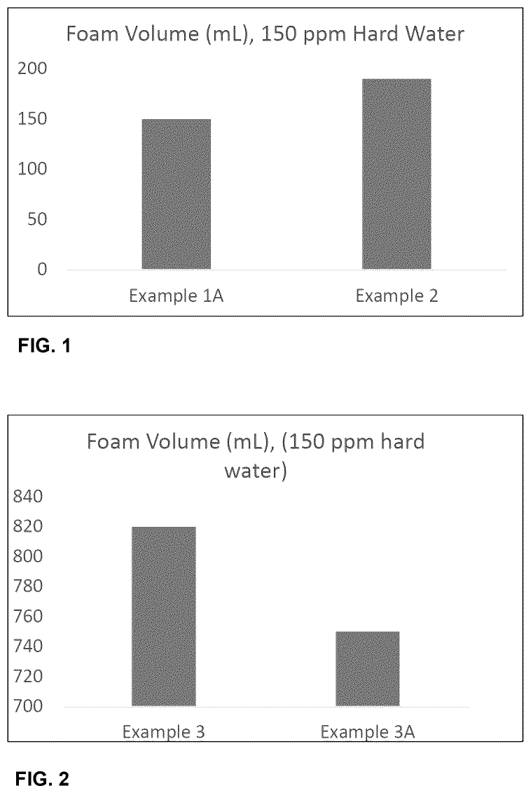

Foaming of compositions of the present invention: FIG. 1 illustrates the foam height of the powder hand wash cleansing compositions of Example 2 and Hand Wash Cleansing Composition of comparative example 1 A. The foam height of the cleansing compositions of present invention (Example 2) is substantially higher than Hand wash composition of prior art GB' 337 (comparative example 1 A).

### Foam Volume:

100 mL of 1% of powder cleansing composition (prepared in 150 ppm water hardness) were used to evaluate the foam volume. The test surfactant solutions were taken in a kitchen blender and mixed at a speed of 2700 rpm for 60 sec. Foam generated was then collected in the 1000 mL measuring cylinder and the foam volume was measured.

The foam volume of test composition was compared graphically as mentioned above. As evident in FIG. 1 Inventive composition of Example 2 exhibits significantly high foam volume than the Comparative Example 1A.

Further, the composition of the present invention shows excellent foam when combined with co-surfactant. Composition of example 3 along with co-surfactants like Galsoft NaLS show foam volume of about 820 mL, while the composition of comparative example 3A, devoid of co-surfactant shows lower foam volume of around 750 mL, as compared to foam volume of composition of example 3, as illustrated in FIG. 2.

### Sensory Test

The various cleansing formulations prepared from powder cleansing compositions of the present invention provide a very good sensory feel to the skin. The Godrej Protekt Mr. Magic Powder composition (Comparative Example 1B) was compared against powder hand wash cleansing composition of present invention (Example 2) for sensory test. FIG. 4 illustrates the results of the sensory test and proves that the hand wash cleansing composition of present invention is significantly better than Godrej's commercially available product.

### Procedure for Sensory Evaluation

i. Test samples were applied on the wet palms in a circular motion generating lather for 15 seconds. The products were rinsed off under a constant flow of water till the time the subject felt that the product had completely rinsed off
ii. Hands were allowed to dry (Towel/Hand Dryer). Test samples were evaluated on different sensorial parameter. The subjects were asked to compare sensory between the test samples subjectively with the help of questionnaire form.

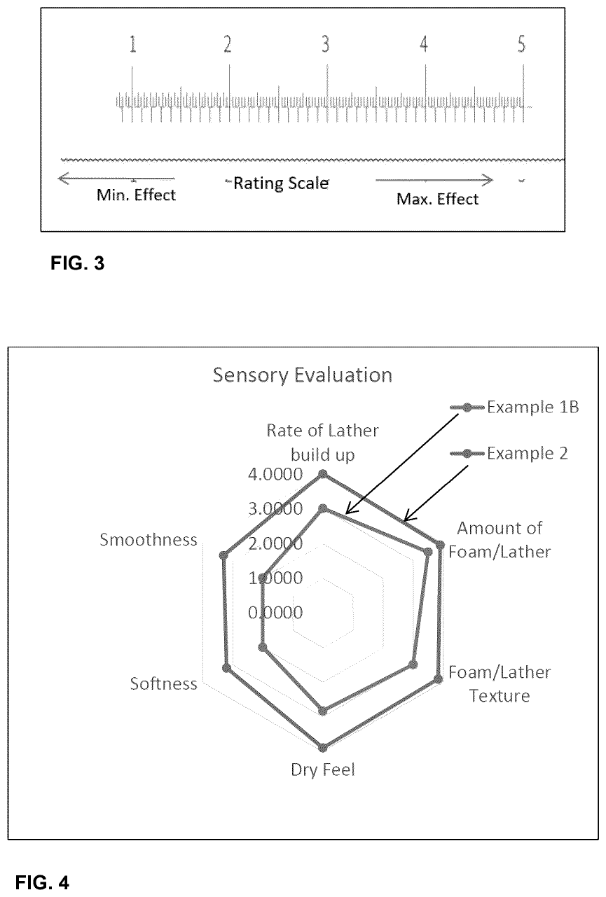

Each test sample was tried in duplicates to ensure repeatability. FIG. 3 shows the rating scale used.

### Moisturization Study

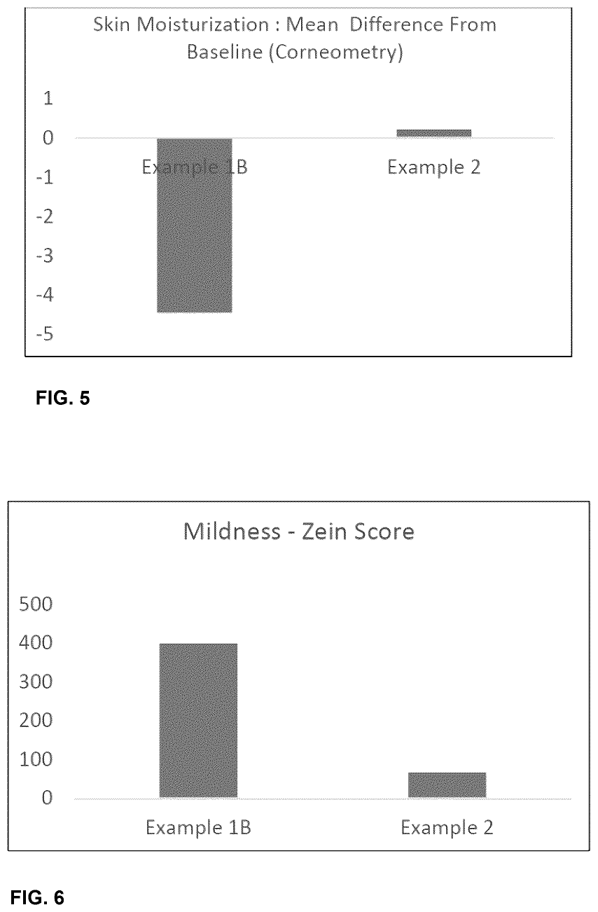

The various skin cleansing formulations prepared from powder cleansing compositions of present invention also improve hydration levels of the skin. The hand wash cleansing composition of Example 2 was compared against Comparative Example 1B for skin moisturization with the help of corneometry. FIG. 5 illustrates the results where hand wash cleansing composition of present invention (example 2) has shown significant improvement in the hydration levels as compared to Godrej Protekt Mr. Magic Powder composition.

### Procedure for Skin Moisturization Study:

Corneometer from Courage Khazaka is used for the study.
1. The Volar forearm is used for the study
2. Each test site is be marked as 4×4 sq. cm on volar forearm
3. Initially baseline reading were measured for each test site
4. Test sample as mentioned above were applied on the respective sites and rinsed as per the protocol
5. Total 15 applications were conducted with a frequency of 2 applications per day
6. Corneometer measurements were conducted after 15 applications
7. The hydration levels were calculated as difference from the baseline readings

### Conditioning Property

The hair-care formulations prepared by diluting powder cleansing composition of the present invention also provides a very good conditioning property to the hair. The hair samples treated with Mild & Moisturizing Shampoo composition prepared using powder cleansing composition of the present invention Example 4 and were compared against untreated hair for delivering conditioning benefit. Hair tresses treated with shampoo composition of example 4 showed good combing force reduction without any tangles during wet and dry combing, feel superior in sensory when dry as compared to untreated hair tresses.

### Zein Score

Various cleansing formulations of the present invention are extremely mild and non-harmful to the skin. Zein (corn derived protein) solubility assay is a commonly used to predict the protein damage potential of the surfactants. The hand wash composition of present invention (Example 2) was compared with comparative example 1B (commercially available Godrej Magic hand wash for Zein score. For Zein protein solubilization assay 1% solution of test samples were used at pH 6.5 to 7.0.

Employing standard protocol of Zein number determination, it has been surprisingly discovered that composition of Example 2 is significantly milder than composition of comparative example 1B, FIG. 6 illustrates the respective zein numbers.

The inventors of present invention have further found that the reconstituted composition show enhanced mildness. Reconstituted powder composition containing N-acyl glutamate and O-acyl isethionate along with co-surfactant show reduced zein score when compared to the reconstituted powder composition containing only N-acyl glutamate and O-acyl isethionates as surfactants (Example 4 and Comparative example 4A). This illustrates synergism between N-acyl glutamate and O-acyl isethionate and co-surfactant as evident from FIG. 7.

1. A mild, natural and sustainable powder cleansing composition comprising blend of N-acyl glutamates and O-acyl isethionates, one or more co-surfactants, suspending agent and organic acid, such that said composition dissolves in water in less than 15 seconds to form a homogenous personal care cleansing formulation.

2. The powder cleansing composition as claimed in claim 1, wherein said blend of N-acyl glutamates and O-acyl isethionates is a blend of N-acyl glutamate of Formula I, and O-acyl isethionates of Formula II 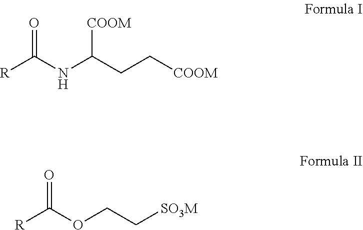
wherein,
R is selected from C₅ to C₂₂ alkyl group, saturated or unsaturated,
M is a cation selected from Li⁺, Na⁺, K⁺, NH₄⁺ and a quaternary ammonium cation derived from tertiary amines.

3. The powder cleansing composition as claimed in claim 2, wherein said composition is prepared by a process comprising steps of:
A) reacting more than one equivalence of fatty acid chloride with alkali metal or ammonium salts of hydroxyalkyl sulphonates to prepare compounds of Formula II,
B) reacting the product of step (A), which contains the remainder fatty acid chloride, with glutamic acid in the presence of a base under typical aqueous Schotten Baumann reaction conditions to form compounds of Formula I; and
C) blending N-acyl glutamate of Formula I, and O-acyl isethionates of Formula II with one or more co-surfactants, suspending agent and organic acid
wherein the pH of the powder cleansing composition is 5.0 to 7.0.

4. The powder cleansing composition as claimed in claim 2, wherein the blend of N-acyl glutamate of Formula I, and O-acyl isethionates of Formula II is present in an amount of 35 to 90% by weight.

5. The powder cleansing composition as claimed in claim 1, wherein one or more co-surfactants are selected from N-acyl glycinate, N-acyl sarcosinate, N-acyl taurate, alkyl amidopropyl sulfobetaines, sulfosuccinates, alkyl betaines, alkyl amidopropyl betaines, and alkyl sulfates.

6. The powder cleansing composition as claimed in claim 5, wherein one or more co-surfactants are present in an amount of 5 to 45% by weight of the total composition.

7. The powder cleansing composition as claimed in claim 1, wherein said organic acid is selected from benzoic acid, dehydroacetic acid and sorbic acid or salt thereof.

8. The powder cleansing composition as claimed in claim 7, wherein said organic acid is present in the amount of 4 to 12% by weight of the total composition.

9. The powder cleansing composition as claimed in claim 1, wherein said suspending agents are selected from hydroxyethylcellulose, hydroxypropylmethyl cellulose, guar gum, gum acacia, gum tragacanth, and xanthan gum.

10. The powder cleansing composition as claimed in claim 9, wherein said suspending agents are present in an amount of 3 to 11% by weight of the total composition.

11. The powder cleansing composition as claimed in claim 1, wherein said composition further comprises fragrance, colour and personal care additives.

12. The powder cleansing composition as claimed in claim 1, wherein said personal care formulation is selected from body wash, face wash, facial foam, hand wash, hand foam, shampoo, shower gel, and shaving foam.
